(19) United States
(12) Patent Application Publication
(10) Pub. No.: US 2013/0011905 A1
(43) Pub. Date: Jan. 10, 2013

(54) INCUBATOR DEVICE AND METHOD OF OPERATING IT

(75) Inventors: Daniel Rapoport, Luebeck (DE); Charli Kruse, Herrnburg (DE); Dennis Wendt, Luebeck (DE)

(73) Assignee: FRAUNHOFER-GESELLSCHSFT ZUR FOERDERUNG ZUR FOERDERUNG DER ANGEWANDTEN FORSCHUNG E.V., Muenchen (DE)

(21) Appl. No.: 13/636,390

(22) PCT Filed: Mar. 23, 2011
(86) PCT No.: PCT/EP2011/001449

§ 371 Date: Sep. 21, 2012

(30) Foreign Application Priority Data
Mar. 25, 2010 (DE) 10 2010 012 790.6

(51) Int. Cl. C12M 1/38; C12N 1/00; C12M 1/04

(52) U.S. Cl. 435/243; 435/303.1; 435/286.6

(57) ABSTRACT

A movable incubator facility comprises a culture chamber which is arranged for receiving at least one cell culture, a heating arrangement by means of which the temperature of the culture chamber can be adjusted, a gas feed arrangement by means of which at least one gas can be introduced into the culture chamber, and an energy storage arrangement which is disposed for supplying the heating arrangement and/or the gas feed arrangement with electrical energy.

The invention relates to an incubator device (also termed a cell culture incubator) for biological materials, particularly for cell cultures and/or tissue cultures.

Setting cultivation conditions for cultivating biological cells with an incubator device and maintaining the same during cultivation is generally known. The standard inventory for cell culture in particular includes a temperable CO₂ incubator which provides and controls the gas atmosphere and the temperature, which are required for the cell culture. It accommodates the cell culture e.g. in multi-well plates, Petri dishes, flasks or the like.

Conventional standard incubators are bulky (approx. 1 m³) and relatively heavy (approx. 100 kg) and also designed for electrical supply via a mains connection. They are therefore not suitable for transporting tasks. For transporting cultivated cells, incubation in a standard incubator has hitherto had to be interrupted. This is disadvantageous however, as the cultivation conditions are changed with transporting, which influences the cells in a non-reproducible and/or uncontrolled manner or even destroys them.

The sending of biological cells in the frozen state, which is known from practice, is not an equivalent alternative to transport under culture conditions, but rather at best a supplement. The freezing/thawing of the cells can lead to the dying off of the cells, the splitting of cell/cell contacts and at least constitutes a substantial disruption of the in vitro culture.

It is the objective of the invention to provide an improved incubator device (cell culture incubator), with which the disadvantages of conventional incubator devices are avoided. The objective of the invention is furthermore to provide an improved method for cultivating biological cells, with which disadvantages of conventional incubation methods are avoided.

This objective is achieved by an incubator device and a method with the features of the independent claims. Advantageous embodiments and applications of the invention result from the dependent claims.

According to a first aspect of the invention, an incubator device is provided, which comprises a culture chamber for receiving at least one cell culture, a temperature control device with which the temperature in the culture chamber can be set, a gas supply device with which at least one gas can be fed into the culture chamber, and an energy storage device which is disposed for supplying the temperature control device and/or the gas supply device with electrical energy. In contrast with conventional incubators, the provision of the energy storage device enables transportability (mobility) of the incubator device whilst it is in an active operating state in which the temperature control device and the gas supply device are switched on and can be actuated. Cultivation conditions can be set whilst the incubator device is separated from stationary supply facilities and in particular is being moved.

The inventors have found that with the components mentioned, a transportable cell incubator is created, which fulfils the following requirements in particular:
(i) small size (smaller than 0.5 m³, particularly smaller than 100 dm³, e.g. smaller than 50 dm³, through to the cm³ range, e.g. approx. 20 cm³) and low mass (less than 50 kg, particularly less than 30 kg, e.g. less than 10 kg, such as 5 kg or less;
(ii) autarkic operation, i.e. operation without an external energy source or media source, particularly for the duration of transporting;
(iii) the cells can be cultivated over a time period of at least 2 hours under standard conditions (for the most part 37° C./5% CO₂) (typical express delivery time); and/or
(iv) according to a variant, the cell incubator can be provided in such a manner that the progression of measured and/or controlled variables (e.g. temperature/CO₂) is logged electronically in order to make it possible to track the process of transporting, which is normally unsupervised.

According to a second aspect of the invention, a method for cultivating biological cells using the incubator device according to the first aspect is provided. To this end, the cells are arranged in the culture chamber of the incubator device and cultivation conditions for the cells in the culture chamber are set.

Further features of preferred embodiments, which may be provided individually or in combination, are summarized in the following.

The energy storage device of the incubator device comprises a primary battery (non-rechargeable) and/or an accumulator (rechargeable). Advantageously, all components of the incubator device can be operated exclusively with the electrical energy which is supplied by the energy storage device. This operation is possible for an operating time which is equal to the typical transport times for transporting cell cultures, e.g. between various medical facilities.

The energy storage device can be equipped with an electrical connecting unit, e.g. plug coupling, in order to temporarily connect the energy storage device to a mains power supply, e.g. public electric power network or on-board electrical system of a vehicle, e.g. transport vehicle and/or laboratory vehicle, e.g. in order to recharge the energy storage device or, in the case of stationary operation, e.g. in a laboratory or in a vehicle, to switch energy supply to the mains power supply.

The culture chamber has a thermally insulated wall, particularly an evacuated or evacuatable wall. The wall may be made e.g. from plastic and/or glass. The culture chamber preferably forms a Dewar flask. Alternatively, the wall may be made from a foam material. Preferably, the thermally insulated wall surrounds the culture chamber on all sides. A part of the thermally insulated wall provided on an upper side of the culture chamber forms a cover, at which the culture chamber can be opened and which can preferably be removed from the culture chamber.

The temperature control device preferably comprises at least one heating device, e.g. with at least one resistance heating element, and/or at least one cooling device, e.g. with at least one Peltier element. Using the temperature control device, a temperature in the range from 10° C. to 40° C. can preferably be set in the culture chamber. The cooling device has particular advantages for the independence of the incubator from external environmental conditions. The coolability allows the use of the incubator also in seasons or climatic zones with high daytime temperatures, e.g. above 30° C.

The incubator device can have a sensor device, particularly at least one temperature sensor and/or at least one gas sensor. At least one sensor (internal sensor) of the sensor device can be arranged in the culture chamber. Alternatively or additionally, at least one sensor (external sensor) of the sensor device is arranged on an external side of the incubator device.

Particularly preferably, the incubator device is equipped with a control circuit (control loop), with which the temperature control device and/or the gas supply device can be controlled depending on signals of the sensor device and at least one predetermined reference variable. The setting of cultivation conditions preferably comprises controlling the temperature control device and/or the gas supply device.

The gas supply device comprises at least one gas source and at least one supply line which leads from the gas source to the culture chamber, wherein the at least one supply line preferably contains a valve with which a gas flow through the supply line can be set.

Advantageously, the at least one gas source can comprise a gas container, particularly a pressurized gas cartridge and/or a gas balloon. The pressurized gas cartridge or the gas balloon may be a replaceable component and separable from the incubator device. Alternatively, the pressurized gas cartridge or the gas balloon may be fixed in the incubator device and refillable.

Typically, the gas supply device is adapted for providing carbon dioxide (CO₂) in the culture chamber. The gas source contains the carbon dioxide. Using the gas supply device, a carbon-dioxide concentration in the range from 1% to 5% can preferably be set in the culture chamber. Alternatively or additionally, further gas-like or steam-like substances can be supplied into the culture space, such as e.g. nitrogen or water vapour (setting humidity).

Alternatively or additionally, a liquid supply device may be provided e.g. for setting the humidity in the culture chamber, which liquid supply device comprises e.g. a water reservoir with a supply line which opens in the culture chamber and is provided with a valve which can be controlled.

Preferably, the incubator device has a control device, using which the temperature control device and/or the gas supply device can be controlled. The control device is preferably connected to the control circuit.

Preferably, the incubator device furthermore has a storage device (data recording system), using which status variables (incubation data), particularly the temperature and/or the gas composition, e.g. the CO₂ content in the culture chamber and/or in the surroundings of the incubator device can be stored electronically (portable autarkic cell incubator with data recording system). The data recording system allows an electronic logging of the incubation data, as a result of which, additional security and traceability of the history of each sample in the incubator are ensured.

A display device may also be provided, which is adapted for displaying operating states of the incubator device and e.g. comprises a display and/or signal lamps. Alternatively or additionally, an alarm device may be provided, e.g. for information about undesired operating states of the incubator device, which alarm comprises e.g. an acoustic signal transmitter and/or signal lamps.

Preferably, a receiving device is provided in the culture chamber for the at least one cell culture, such as e.g. a mount for at least one multi-well plate, at least one culture flask and/or at least one Petri dish.

Preferably, the incubator device has a container (case, box, cage), in which the culture chamber, the temperature control device, the gas supply device and the energy storage device are accommodated together. A particularly simple structure results if the container has the culture chamber and additionally a control chamber in which the temperature control device, the gas supply device and the energy storage device are accommodated.

The container can be equipped with a container lid. The container lid can be opened in order to enable access e.g. to the culture chamber and/or the other components of the incubator device. For a simplified inspection of the operating state of the incubator device, the container cover can be produced at least to some extent from a transparent material, e.g. plastic or glass.

Advantageously, the incubator device can be operated independently of and separately from permanently installed supply lines which are provided for connection to stationary devices. Cultivation conditions can be set and/or maintained in an autarkic manner for a duration of at least 2 hours, preferably at least 8 hours, particularly preferably at least 12 hours, such as 48 hours. The incubator device can be constructed in such a compact manner that it can be moved manually, particularly carried.

Further details and advantages of the invention will be described below with reference to the attached drawing. The FIGURE shows as follows:

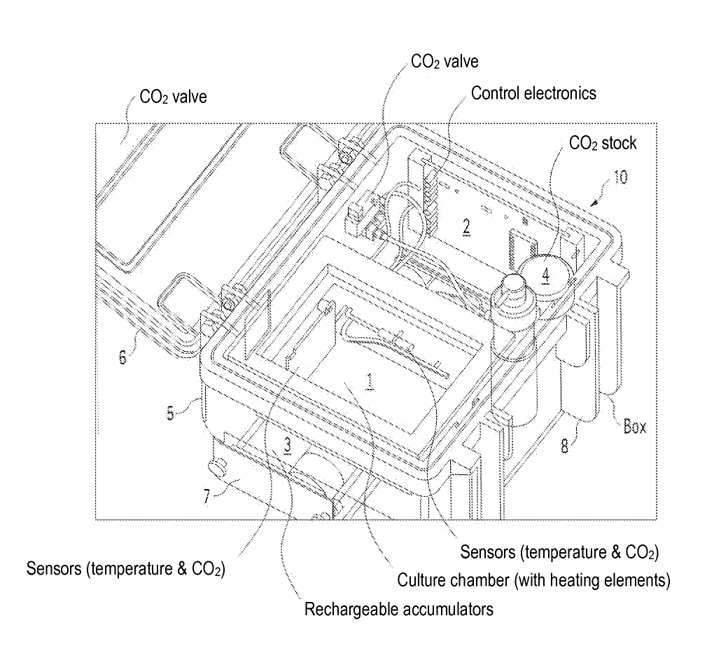

FIG. 1: a schematic perspective view of a preferred embodiment of the incubator device according to the invention.

FIG. 1 illustrates a principal structure of the transportable cell culture incubator with the important functional elements indicated. The portable incubator device (cell culture incubator) 10 comprises a closable box (container 5 with an upper container cover 6 and a side container lid 7) which is divided into a culture chamber 1 and a control chamber 2. The culture chamber 1 is surrounded by thermally insulating walls, in order to maintain the temperature constancy inside the culture chamber 1 and to minimize the energy consumption of the incubator.

A heating element and sensors (temperature and CO₂), with the aid of which the culture conditions are monitored, are located in the culture chamber 1. An adapted receiving device (not illustrated) for the respective cell culture vessel (multi-well plate, Petri dish, flask, etc.) is located in the culture chamber 1. The control chamber 2 accommodates an energy source 3 (energy storage device, e.g. rechargeable accumulator), a gas supply device 4 with a CO₂ source (e.g. commercially available CO₂ cartridge) and connecting lines to a valve and from the same into the culture chamber. Electronic and/or electromechanical elements (control device) are also provided, with the aid of which the temperature and also the CO₂ content in the culture chamber 1 are controlled. In addition, the incubator has a data recording system integrated in the control electronics (part of the control device), which system stores the progression of the controlled variables and thus makes it possible to log the often unsupervised transport process.

The side container cover 7 allows access to the energy source 3 without opening the upper container lid 6, e.g. in order to exchange the accumulators.

The container 5 has lateral profiles 8 of the container wall, with which the container wall is stabilized (and therefore the cell culture is protected). The side profiles are preferably formed complementarily to one another on mutually opposite sides of the container 5, so that stable stacking or assembly of a plurality of containers is facilitated. Furthermore, handles (not illustrated) can be provided, using which the container 5 can be conveyed manually.

The features of the invention disclosed in the previous description, the drawings and the claims can be significant individually as well as in combination for the realization of the invention in its different embodiments.

1-20. (canceled)

21. An incubator device, comprising:
a culture chamber, which is adapted for receiving at least one cell culture;
a temperature control device with which a temperature in the culture chamber can be set;
a gas supply device with which at least one gas can be introduced into the culture chamber; and
an energy storage device, which is arranged for supplying at least one of the temperature control device and the gas supply device with electrical energy.

22. The incubator device according to claim 21, wherein the energy storage device comprises at least one of a primary battery and an accumulator.

23. The incubator device according to claim 21, wherein the culture chamber has a thermally insulated wall.

24. The incubator device according to claim 23, wherein the thermally insulated wall surrounds the culture chamber on all sides.

25. The incubator device according to claim 23, wherein the thermally insulated wall comprises a lid on an upper side of the culture chamber.

26. The incubator device according to claim 21, wherein the temperature control device comprises at least one of a heating device and a cooling device.

27. The incubator device according to claim 26, wherein the temperature control device comprises at least one of at least one resistance heating element and at least one Peltier element.

28. The incubator device according to claim 21, which further comprises a sensor device.

29. The incubator device according to claim 28, wherein the sensor device comprises at least one of at least one temperature sensor and at least one gas sensor.

30. The incubator device according to claim 28, comprising at least one feature selected from the group consisting of:
at least one sensor of the sensor device is arranged in the culture chamber,
at least one sensor of the sensor device is arranged on an external side of the incubator device, and
a control loop is provided, in which at least one of the temperature control device and the gas supply device can be controlled as a function of signals of the sensor device and at least one predetermined reference variable.

31. The incubator device according to claim 21, wherein the gas supply device comprises at least one gas source and at least one supply line which leads from the gas source to the culture chamber, wherein the at least one supply line optionally contains a valve with which a gas flow through the supply line can be set.

32. The incubator device according to claim 31, wherein the at least one gas source comprises a gas container.

33. The incubator device according to claim 32, wherein the gas container comprises at least one of a pressurized gas cartridge and a gas balloon.

34. The incubator device according to claim 33, wherein the pressurized gas cartridge or the gas balloon is a replaceable component and is separable from the incubator device or is fixed in the incubator device and refillable.

35. The incubator device according to claim 21, wherein the gas supply device is adapted for providing carbon dioxide (CO₂) in the culture chamber.

36. The incubator device according to claim 21, which further comprises a control device, using which at least one of the temperature control device and the gas supply device can be controlled.

37. The incubator device according to claim 21, which further comprises at least one of
a storage device, using which status variables in the culture chamber of the incubator device can be stored electronically, and
a display device which is adapted for displaying operating states of the incubator device.

38. The incubator device according to claim 37, wherein the storage device is a data recording system.

39. The incubator device according to claim 37, wherein the storage device is adapted for electronically storing at least one of the temperature and the gas composition in the culture chamber of the incubator device.

40. The incubator device according to claim 39, wherein the storage device is adapted for electronically storing at least one of the temperature and the CO₂ content in the culture chamber of the incubator device.

41. The incubator device according to claim 21, wherein a receiving device is provided in the culture chamber for the at least one cell culture.

42. The incubator device according to claim 21, wherein the receiving device comprises a mount for at least one of at least one multi-well plate, at least one culture flask and at least one Petri dish.

43. The incubator device according to claim 21, wherein the culture chamber, the temperature control device, the gas supply device and the energy storage device are accommodated together in a container with an at least partially transparent upper container lid.

44. The incubator device according to claim 21, comprising at least one feature selected from the group consisting of:
the incubator device can be operated independently of permanently installed supply lines for stationary devices,
the incubator device can be moved manually, and
the incubator device is set up for at least one of automatic setting and maintenance of cultivation conditions over at least 2 hours.

45. The incubator device according to claim 21, which can be carried by hand.

46. A method for cultivating biological cells using an incubator device according to claim 21, said method comprising the steps of:
providing the cells in the culture chamber of the incubator device, and
setting cultivation conditions for the cells.

47. The method according to claim 46, wherein the setting of cultivation conditions comprises at least one of controlling the temperature control device and the gas supply device.
